(19) United States
(12) Patent Application Publication
(10) Pub. No.: US 2019/0080795 A1
(43) Pub. Date: Mar. 14, 2019

(54) DIGITIZED NEEDLE BIOPSIES NUCLEI INFORMATION BASED PROSTATE CANCER DETECTION, GRADING, SCORING, AND REPORTING SYSTEMS AND METHODS

(71) Applicants: Foram Mahendra Sanghavi, San Antonio, TX (US); Sos S. Agaian, San Antonio, TX (US)

(72) Inventors: Foram Mahendra Sanghavi, San Antonio, TX (US); Sos S. Agaian, San Antonio, TX (US)

(21) Appl. No.: 16/132,341

(22) Filed: Sep. 14, 2018

Related U.S. Application Data
(60) Provisional application No. 62/558,455, filed on Sep. 14, 2017.

(51) Int. Cl. G16H 30/40; G16H 50/20; G06T 7/00; G06K 9/00

(52) U.S. Cl. CPC G16H 30/40; G16H 50/20; G06T 2207/30081; G06K 9/0014; G06T 7/0012

(57) ABSTRACT

A method and system for evaluating prostate cancer needle biopsy images for cancer using the nuclei information. The various embodiments of the present invention include the different methods which could be used distinctly or in unification to automatically diagnose, classify, and report the results for the prostate cancer needle biopsy images. Different feature sets are targeted for distinct methods and system, for instance, structural, textural, and geometrical features are utilized for cancer detection, morphological features for Gleason scoring. The invention provides a system to generate the cancer aggressive maps for the whole slide needle biopsy images to help detect the malignancy of prostate cancer. The invention provides the system to perform N-scoring on the prostate cancer tissue samples. The invention also provides the system to generate pathology report automatically.

### PRIORITY CLAIM

This application claims priority to U.S. Provisional Application Ser. No. 62/558,455 entitled “Digitized Needle Biopsies Nuclei Information Based Prostate Cancer Detection, Grading, Scoring, And Reporting Systems And Methods.” filed Sep. 14, 2017, which is incorporated herein by reference in its entirety.

### STATEMENT REGARDING FEDERALLY SPONSORED RESEARCH OR DEVELOPMENT

None.

### BACKGROUND OF THE INVENTION

### 1. Field of the Invention

The present invention(s) relate to relates to digitized automated image processing of digitized prostate needle biopsy images, and utilization nuclei information for cancer diagnosis, cancer grading or N-Gleason scoring, cancer aggressive map generation and automated report formation.

### 2. Description of the Relevant Art

Prostate cancer is a major health issue in the United States as well as in the World. Prostate Cancer is a common type of cancer after Skin cancer and is a second primary cause of death after lung cancer in the American men. The most common scoring system utilized for detecting prostate cancer is the Gleason Grading system. The pathologists, evaluate the biopsy tissue samples using this scoring system and can be variable, even by the same pathologists. The accuracy and effectiveness of the pathologist's reports is affected by various factors such as training, skill, tiredness, individual interpretation, and application of Gleason grading to only small portions of prostate tissue (i.e. biopsy samples). To support and improve the efficiency and accuracy of the pathologist's analysis, there is a need for an automated Gleason grading and scoring system.

Various classification systems based on texture, and tissue-components (nuclei, glands, stroma, etc.) have been employed to classify prostate histopathology images. However, very few researches have been done on using nuclei information for cancer detection and classification of prostate biopsy images.

U.S. Patent Application No. 2010/0098306 AI to Madhubashi et al. relates to a method or system using content-based image retrieval of the biopsy image features based on pre-determined criteria and their analysis to diagnose cancer. The method distinguishes the section as cancerous, non-cancerous and uncertain. The system has the following steps: (1) Reading the input biopsy image, (2) Identifying the suspected region, (3) Feature extraction, (4) reduction in feature dimensionality, and (5) classification of the identified region and reclassification at a higher magnification. The features extracted in this system are, first and second order statistics of color channels, graphical features (Voronoi, Delaunay and MST from nuclei centers. The features extracted from the graphs are: standard deviation, average, minimum to maximum ratio, entropy of edges and areas of polygons and triangles, statistics of Co-adjacency matrix constructed from the glands centers), textural features such as Haar wavelet feature and Gabor filter response, Sobel, derivatives and Kirsch filter response and morphological information (nuclear density and gland morphology). The accuracy of 92.8% is reported for distinguishing the Grade 3 cancer from the stroma but for classifying grade 3 vs 4 the accuracy is 76.9%.

Jain Anil et al. proposes a system for identifying prostate cancer using the combination of nuclear features and textural features in Nguyen, K., Jain, A. K., & Sabata, B. (2011). Prostate cancer detection: Fusion of cytological and textural features. Journal of pathology informatics, 2(2), 3. On experimentation, the fusion of cytological and textural features reported the true positive rate of 78% for identifying cancer regions.

In the study, Contributions to computer-aided diagnosis of prostate cancer in histopathology (Doctoral dissertation, Michigan State University) by Nguyen, K. (2013) combined texture based features with nucleoli in nucleus features to classify normal images vs grade 3, normal images vs grade 4, and grade 3 vs grade 4 images reporting accuracies of 97.4%, 99.1% and 83% respectively.

However, from the above studies, it can be noted that the nuclei information is used in combination with glandular information for prostate cancer detection and classification. Thus, conceiving a need of the complete nuclei information based automated system for diagnosis and classification of prostate cancer needle biopsy images.

Studies have shown that cell and alteration in its structure, provide useful information for diagnosing the type of disease and its aggressiveness. Changes in particular cell type helps identifying a particular type of disease. For example, to identify and grade hepatocellular carcinoma the neoplastic liver cells are observed, similarly, lymphocytes are studied for detecting the presence of viral hepatitis etc. [1]. Modification in cellular structure, particularly nuclei, is primarily used for diagnosing the cancer cell from the non-cancer cell for different types of cancers in the given tissue region [1-3]. The alteration in the size, shape and the chromatin texture help distinguish the tumor nuclei from the normal nuclei.

Nuclear enlargement, pleomorphism, prominence of nucleoli, crowding, etc. are some the significant nuclear features for detecting prostate cancer. The nucleolar enlargement predominantly from nuclei enlargement, plays an important role in forming a decisive criterion for the presence cancer. The nuclei with area greater than 60 μm² are diagnosed as cancerous and nuclei with area less than 35 μm² as non-cancerous. Detection of large nucleoli in the secretory epithelium cells play a very important role in diagnosing cancer in prostate adenocarcinoma. Larger the nucleoli, more is the probability of the sampled tissue to be cancerous with higher grade. However, there are chances of prominent nucleoli to be present in the non-cancerous tissue region or to be of smaller size or absent in the cancer region. Therefore, making them not the perfect markers for cancer detection but can be still used for detection purposes along with other features [4].

The following is the advantage of analyzing the nuclear especially, the nucleolar morphometry, that they help in predicting the degree of progress of the cancer, the metastasis, and biochemical reoccurrence of the cancer. The authors in this study observed, a direct correlation between the Gleason grading and the presence of nucleoli [3]. Thus, it is important to target nuclei information in the histopathology images of prostate cancer to achieve a more accurate detection and grading of prostate cancer.

Various segmentation approaches have been developed in the past for nuclei segmentation in the biopsy images, [Irshad, H., Veillard, A., Roux, L., & Racoceanu, D. (2014). Methods for nuclei detection, segmentation, and classification in digital needle biopsy: a review—current status and future potential. IEEE reviews in biomedical engineering, 7, 97-114] but there still persists the problem of segmenting the overlapping nuclei. Consequently, there is a need of a more robust nuclei segmentation approach. Feature extraction play an aiding step for the classifying the prostate cancer needle biopsy images. Although, different features based on texture, morphology and texture have been proposed and used in the past, new features are required along with existing features to help improve the analysis of the prostate biopsy tissue sample. Employment of an appropriate classifier is a key step for classification. Using a single classifier sometimes do not work accurately for classifying the different grades of cancer. Thus, to improve the efficiency of the classification, multi-classifier ensemble is designed.

Secondary Gleason scoring system is utilized currently for classifying the prostate cancer images. Many researches have been conducted generating automated Gleason score, however, there are very few to none studies reporting the Gleason scoring using nuclei information. Cancer map is a visual representation of the areas of cancer in the whole slide biopsy samples. Very few researches have automated cancer map generation, but none of them are based on complete nuclei information thus making it as a requirement.

[Agaian, S. S., Lopez, C. M. M., Almuntashri, A., & Metzler, R. (2012). U.S. patent application Ser. No. 14/347,912.]

Pathological report is generated by the pathologist by manually inputting the diagnosis information in the report comprising the patient information, which is a tedious job and could be error prone. To help solve this problem, we need an automated report generation system.

Thus, we need an automated system that analyzes the prostate cancer biopsy sample using nuclei information for cancer detection, grading and scoring and for generating the automated pathological report.

### SUMMARY OF THE INVENTION

The present invention(s) include systems, methods, and apparatuses for, or for use in: (i) diagnosing and classifying Prostate Cancer Needle biopsy Images; (ii) shape based segmenting of the nuclei information from the images; (iii) Human Visual System based visualization.

The Summary introduces key concepts related to the present invention(s). However, the description, figures, and images included herein are not intended to be used as an aid to determine the scope of the claimed subject matter. Moreover, the Summary is not intended to limit the scope of the invention.

In some embodiments, the present invention(s) provide a system and a method for diagnosing and classifying the Prostate Cancer Needle biopsy Images comprising:

collecting training data and testing data, wherein testing data has whole slide images and magnified images; segmenting the nuclei information from the other tissue components using the alpha-trimmed mean; generating feature vector using the nuclei information to diagnose cancer in the prostate needle biopsy images; classifying the obtained feature vector through the decision tree classifier to diagnose cancer; generating visual dictionary of nuclei images only if the images are detected cancerous; extracting features using the visual dictionary and generating feature vector; classifying the cancer images into their respective grades using the decision tree classifier.

In some embodiments, the present invention(s) allows for a method for shape based segmenting of the nuclei information from the images comprising:

dividing the images into block of the size of the training images if the images are whole slide images or else proceed to next step of image enhancement; performing image enhancement; selecting a color model and separating the image into different color planes of the selected model;

performing segmentation using threshold (T) based on Alpha-trimmed mean on each color planes using the following formulae.

$$t = \frac{1}{MN} {\sum }_{i = 0}^{M - 1} {\sum }_{j = 0}^{N - 1} [ ( \frac{a}{a + b} ) I ( i , j ) - ( \frac{b}{a + b} ) α ( i , j ) ]$$

$$T = max ( ∝ ) + t$$

Where,

α is the alpha-trimmed mean image of the image I,

I is the color plane of the image,

a, b is the constants,

M, N is the size of the image,

t is the mathematical function calculating weighted difference between the image I and α-trimmed mean image for each pixel, and

T is the threshold value calculated by taking sum of the maximum value of a trimmed mean and t.

And fusing the obtained segmented images to form a color segmented image.

In some embodiments, the present invention(s) allows for a method for diagnosing cancerous and non-cancerous prostate needle biopsy images comprising:

reading the nuclei segmented images of training and testing database; extracting nuclei based structural/textural features using Human Visual System based visualization; extracting nuclei based geometrical features using graphical methods; generating feature vector on combining features obtained and performing feature selection; training the decision tree classifier and classifying the test data set using the trained classifier as either cancerous or non-cancerous.

In some embodiments, the present invention(s) allows for a method for Human Visual System based visualization comprising:

reading the shape based nuclei segmented image; generating weber image using the modified weber law giving by the formula:

$$w = \frac{1}{( \frac{N}{2} ) + 1} {\sum }_{i = 0}^{( \frac{N}{2} ) - 1} \frac{\vert {n}_{t} - {n}_{c} {\vert}^{γ}}{{n}_{t} + {n}_{c}} , t = 2 * i + 1$$

Where,

N is the number of neighbors selected,

γ is the power constant,

w is the weber coefficient for block of size M×M,

nₜ is the calculated t^{th} pixel of the block, and

n_{c} is the center pixel of the M×M block.

Then selecting a N×N block from the weber image and dividing into sub-blocks of size m×m; analyzing the horizontally and vertically aligned sub-blocks with respect to center sub-block; generating a 3×3 block such that above obtained analyzed value is stored in their corresponding locations and diagonal values as those obtained at radius r from the center pixel in the center block; generating the nuclei-structured based Fibonacci p-patterns using the following mathematical equation:

$$F = {\sum }_{k = 0}^{\frac{n}{2} - 1} {b}_{k} {a}_{2 * k + 1} {f}_{p} ( k ) + {\sum }_{k = 0}^{\frac{n}{2} - 1} {c}_{k} {a}_{2 * k} {f}_{p} ( k )$$

$$Where , \\ {a}_{2 * k + 1} = \{ \begin{matrix}1 , & {X}_{k} - {X}_{c} \geq t 1 \\ 0 , & {X}_{k} - {X}_{c} < t 1\end{matrix} , where k = 0 , … , \frac{n}{2} - 1 \\ {a}_{2 * k} = \{ \begin{matrix}1 , & {X}_{k} - {X}_{c} \geq t 2 \\ 0 , & {X}_{k} - {X}_{c} < t 2\end{matrix} , where k = 0 , … , \frac{n}{2} - 1 \\ {f}_{p} ( k ) = \{ \begin{matrix}0 , & k < 0 \\ 1 , & k = 0 \\ {f}_{p} ( k - 1 ) + {f}_{p} ( k - p - 1 ) , & k > 0\end{matrix}$$

bₖ, cₖ are the weights assigned to corresponding values of αₖ,
F is the generated Fibonacci p-patterns generated of each radius,
n is the number neighbors selected here n=8,
Xₖ is the pixel value at k^{th} place in the formed 3×3 block,
X_{c} is the center pixel of the N×N block,

t1 and t2 are the threshold values, and

αₖ, is the Fibonacci image.

In some embodiments, the present invention(s) allows for a method for identifying the grade of cancer for the detected cancerous prostate needle biopsy images comprising

Reading the nuclei segmented images of training and testing set; generating manually the nuclei database for different grades of cancer; generating dictionary of visual words using the automated and manually generated nuclei segmented training set of images; generating feature vector using the effective number of visual dictionary features obtained in the human interactive nuclei based Bag-of-Words approach; applying classifier tree to classify the testing set of cancerous needle biopsy images to its respective grade of cancer i.e. Grade 3, 4 or 5.

In some embodiments, the present invention(s) allows for a method of decision tree based classifier ensemble comprising:

Reading the feature vector-1 generated from the nuclei based structural and geometrical features;

applying classifier 1 for detecting cancerous and non-cancerous prostate needle biopsy images;

reading feature vector 2 generated from the nuclei based bag-of-words approach;

applying classifier 2 for classifying the Gleason grade 3 prostate cancer needle biopsy images from grade 4 and 5; reading feature vector 3 generated from the nuclei based bag-of-words approach; applying classifier 3 for classifying the Gleason grade 4 prostate cancer needle biopsy images from grade 5.

In some embodiments, the present invention(s) allows for a method for Gleason scoring the prostate cancer needle biopsy images comprising:

generating nuclei database for each Gleason grade 3,4 and 5; reading the testing set of images;

performing nuclei segmentation on the testing images; extracting nuclear morphometric features from both, the nuclei database and testing images; comparing the segmented nuclei features with the generated database features; assigning primary grading if the nuclei in the testing image belong to one grade of cancer; calculating the percentage of grades present in the testing image if there are more grades present based on nuclei grade similarity; assigning secondary scoring if percentage similarity of two grades are present in the testing images or else ternary or n-scoring if 3 or more grades are counted for testing image.

In some embodiments, the present invention(s) allows for a method of generating a cancer map using the cancer aggressiveness measurement comprising: reading the test whole slide images and train magnified set of images; performing nuclei segmentation on both training and testing data set images; calculating the cancer aggressiveness measurement for each block of the segmented whole slide image; calculating the average cancer aggressiveness measurement for grade of cancer from the training images; assigning the block value 1 if the value of cancer aggressiveness measurement for testing block is greater or equal then training block or else 0; generating cancer map from the above step.

In some embodiments, the present invention(s) allows for a method of automated report generation comprising: retrieving the patient information; reading the different sections of the biopsy sample; generating the prostate map by recording the evaluated output of the biopsy sample sections at their corresponding location; automated writing of the diagnosis of the patients' output for each biopsy section of the sample and cumulative diagnosis of the sample.

In some embodiments, techniques or applications are fully automated and are performed by a computing device, such as, for example, a central processing unit (CPU), graphics processing unit (GPU), field programmable gate array (FPGA), and/or application specific integrated circuit (ASIC).

### BRIEF DESCRIPTION OF THE DRAWINGS

Advantages of the present invention will become apparent to those skilled in the art with the benefit of the following detailed description of embodiments and upon reference to the accompanying drawings in which:

FIG. 1 shows the flow diagram of the Prostate Needle biopsy image cancer diagnosis and classification system according to the embodiment of the present invention.

FIG. 2 shows the flow diagram of the Nuclei Segmentation process according to the embodiment of the present invention.

FIG. 3 shows the flow diagram for prostate cancer diagnosis according to the embodiment of the present invention.

FIG. 4 shows the flow diagram for classifying prostate cancer needle biopsy images into different grades of cancer according to the embodiment of the present invention.

FIG. 5 shows the flow diagram of the decision tree classifier ensemble according to the embodiment of the present invention.

FIG. 6 shows the flow diagram of the Gleason scoring approach for the prostate cancer needle biopsy images using nuclei morphometric features according to the embodiment of the present invention.

FIG. 7 shows the flow diagram of Cancer Map generation using the cancer aggressive measurement features according to the embodiment of the present invention.

FIG. 8 shows an example of normal and grade 3 needle biopsy images according to the embodiment of the present invention.

FIG. 9 shows an example of grade 4 and 5 needle biopsy images according to the embodiment of the present invention.

FIG. 10 shows an example of whole slide needle biopsy images of different race according to the embodiment of the present invention.

FIG. 11 shows illustrative example of shape based nuclei segmentation for normal and Grade 3 needle biopsy images according to the embodiment of the present invention.

FIG. 12 shows illustrative example of shape based nuclei segmentation for Grade 4 and 5 needle biopsy images according to the embodiment of the present invention.

FIG. 13 shows illustrative example of shape based nuclei segmentation for whole slide needle biopsy image of American African man according to the embodiment of the present invention.

FIG. 14 shows illustrative example of shape based nuclei segmentation for whole slide needle biopsy image of Hispanic man according to the embodiment of the present invention.

FIG. 15 shows illustrative example of shape based nuclei segmentation for whole slide needle biopsy image of White man according to the embodiment of the present invention.

FIG. 16A-C shows illustrative examples of nucleus from prostate needle biopsy images of grade 3, 4 and 5 according to the embodiment of the present invention.

FIG. 17 shows the illustrative example of weber image using different sizes according to the embodiment of the present invention.

FIG. 18 shows block diagram of the automated report generating system according to the embodiment of the present invention.

While the invention may be susceptible to various modifications and alternative forms, specific embodiments thereof are shown by way of example in the drawings and will herein be described in detail. The drawings may not be to scale. It should be understood, however, that the drawings and detailed description thereto are not intended to limit the invention to the particular form disclosed, but to the contrary, the intention is to cover all modifications, equivalents, and alternatives falling within the spirit and scope of the present invention as defined by the appended claims.

### DETAILED DESCRIPTION OF THE PREFERRED EMBODIMENTS

It is to be understood the present invention is not limited to particular devices or methods, which may, of course, vary. It is also to be understood that the terminology used herein is for the purpose of describing particular embodiments only, and is not intended to be limiting. As used in this specification and the appended claims, the singular forms “a”, “an”, and “the” include singular and plural referents unless the content clearly dictates otherwise. Furthermore, the word “may” is used throughout this application in a permissive sense (i.e., having the potential to, being able to), not in a mandatory sense (i.e., must). The term “include,” and derivations thereof, mean “including, but not limited to.” The term “coupled” means directly or indirectly connected.

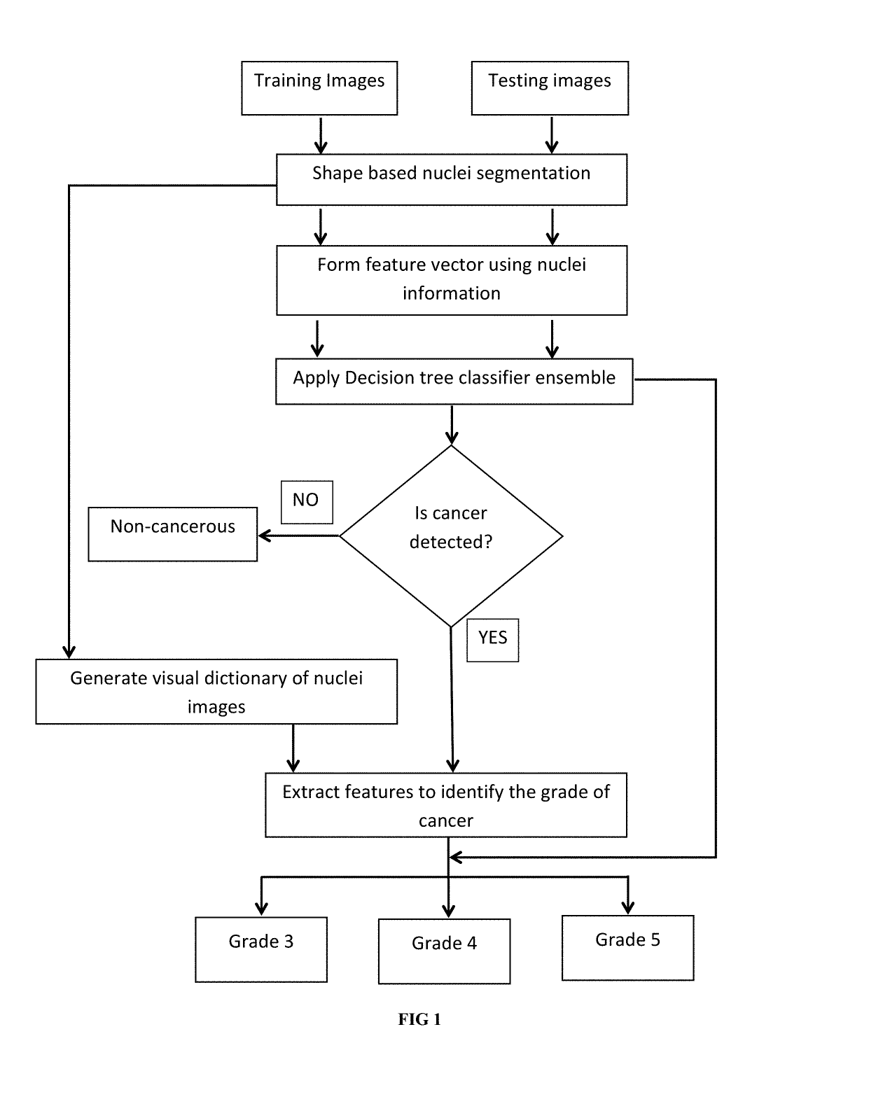

The process according to the embodiment of the present invention for computer aided detection and classification of the prostate needle biopsy images using nuclei information shown in FIG. 1 is described below. The training and testing set of images are read as input, wherein the training set includes magnified prostate needle biopsy images and testing images comprise of magnified and whole slide images of prostate needle biopsy. Shape based nuclei segmentation is performed on the read training and testing set of images acquiring the nuclei information from the images. Features extraction is performed on the segmented nuclei information, generating a feature vector which is further used for classification either as cancerous or non-cancerous. If the image is diagnosed as cancerous, nuclei features using the visual dictionary of nuclei images are extracted to identify the grade of cancer. The visual dictionary of nuclei images of different grades of cancer is generated from the segmented cancerous training images. The decision tree classifier ensemble is applied on the extracted features to classify the prostate needle biopsy images as both cancerous or non-cancerous and the classified cancerous images into Gleason grade 3, 4 or 5.

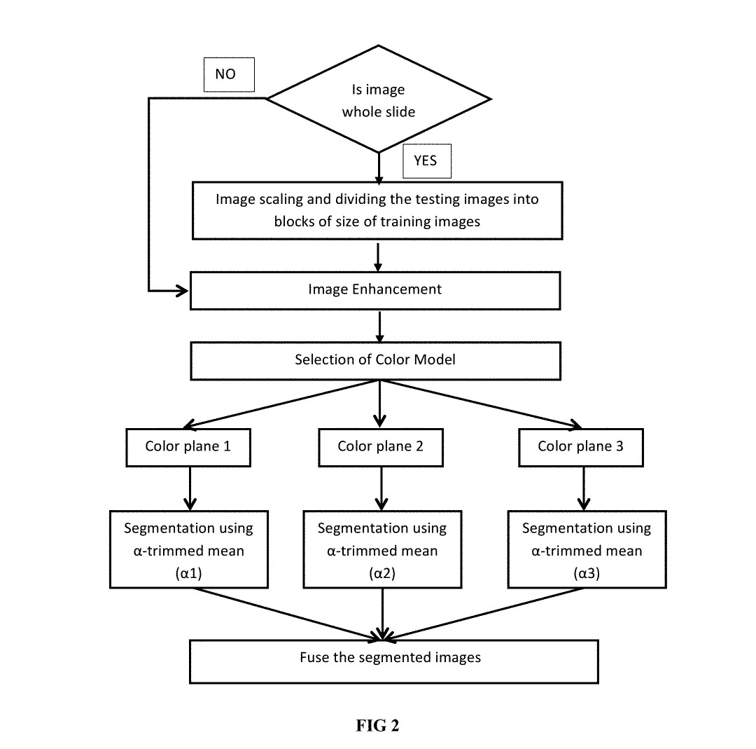

The process according to the embodiment of the present invention for shape based nuclei segmentation shown in FIG. 2 is described below. Prior to image enhancement, image scaling is performed if the images are whole slide images. The scaled images are divided into blocks of the size of the training images. The block images of whole slide image or the magnified images are enhanced using color image enhancement method depending on the color contrast of the image.

A color model is selected for the images and the color conversion is performed when required. The new color image is segregated into different color planes. Segmentation using α-trimmed mean is performed on each color plane. Thresholding method is used for segmentation purpose in the embodiment with the threshold value calculated using the following formula:

$$\begin{matrix}t = \frac{1}{MN} {\sum }_{i = 0}^{M - 1} {\sum }_{j = 0}^{N - 1} [ ( \frac{a}{a + b} ) I ( i , j ) - ( \frac{b}{a + b} ) α ( i , j ) ] & ( 1 ) \\ T = max ( α ) + t & ( 2 )\end{matrix}$$

Where,

α is the alpha-trimmed mean image of the image I referred from, Chernenko, S. Alpha-trimmed mean filter—Librow—software. Retrieved Sep. 7, 2016, http://www.librow.com/articles/article-7,

I is the color plane of the image,

a, b is the constants,

M, N is the size of the image,

t is the mathematical function calculating weighted difference between the image I and α-trimmed mean image for each pixel, and

T is the threshold value calculated by taking sum of the maximum value of a trimmed mean and t.

Thresholding is performed using the following definition:

$${I}_{—} thresh ( i , j ) = \{ \begin{matrix}I ( i , j ) , & x \leq T \\ 0 , & x > T\end{matrix} ∀ i , j \qquad ( 3 )$$

Where T is the threshold value for thresholding the image I, and

I_thresh is the binarized output obtained on thresholding.

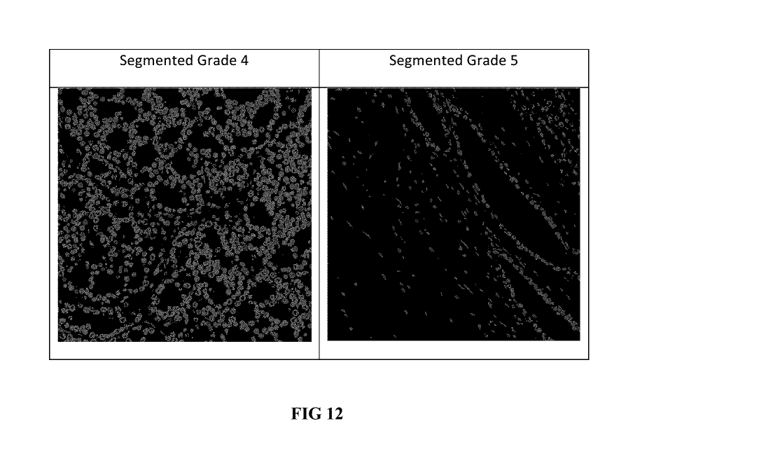

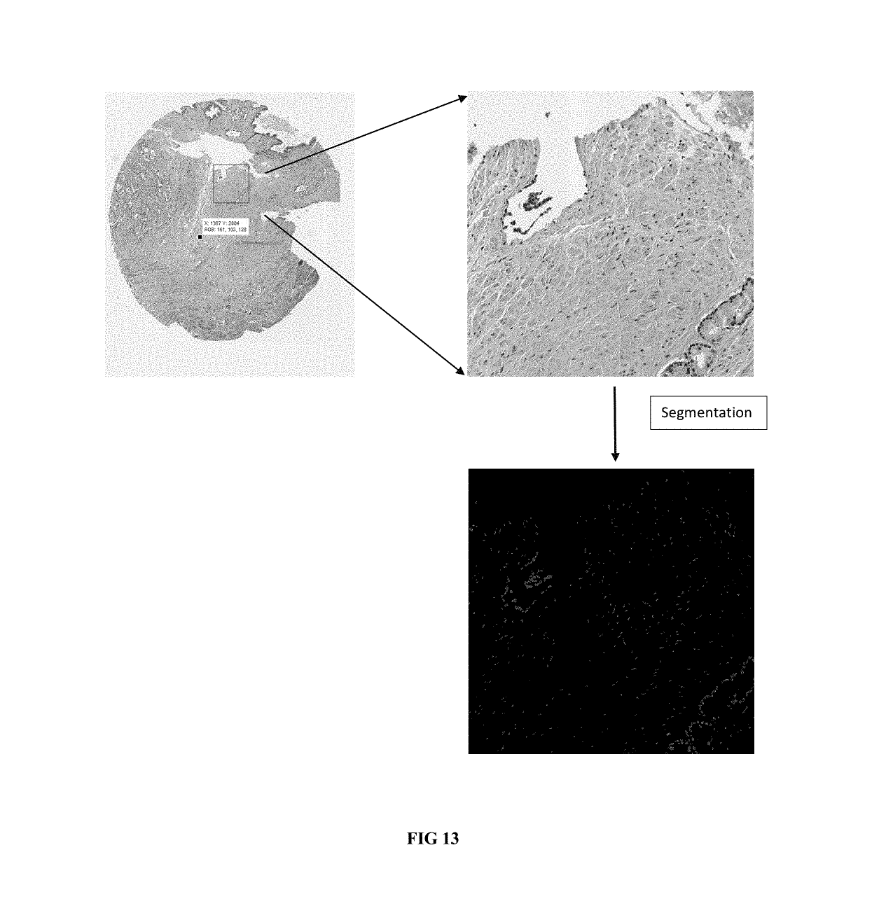

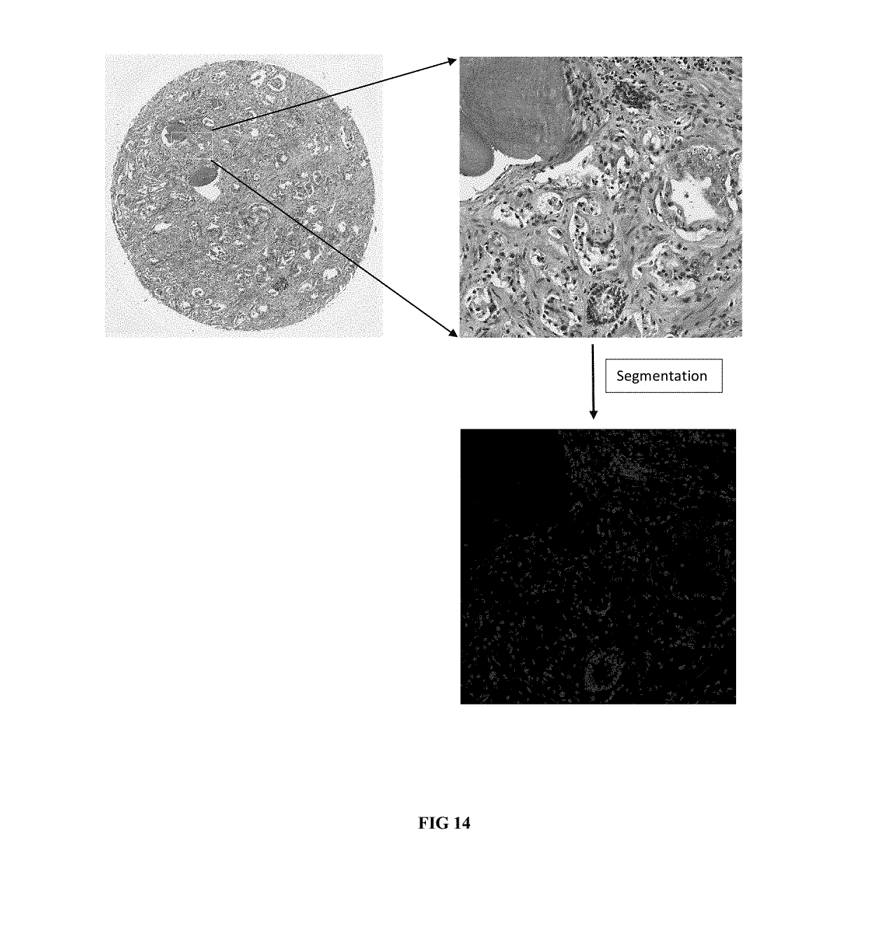

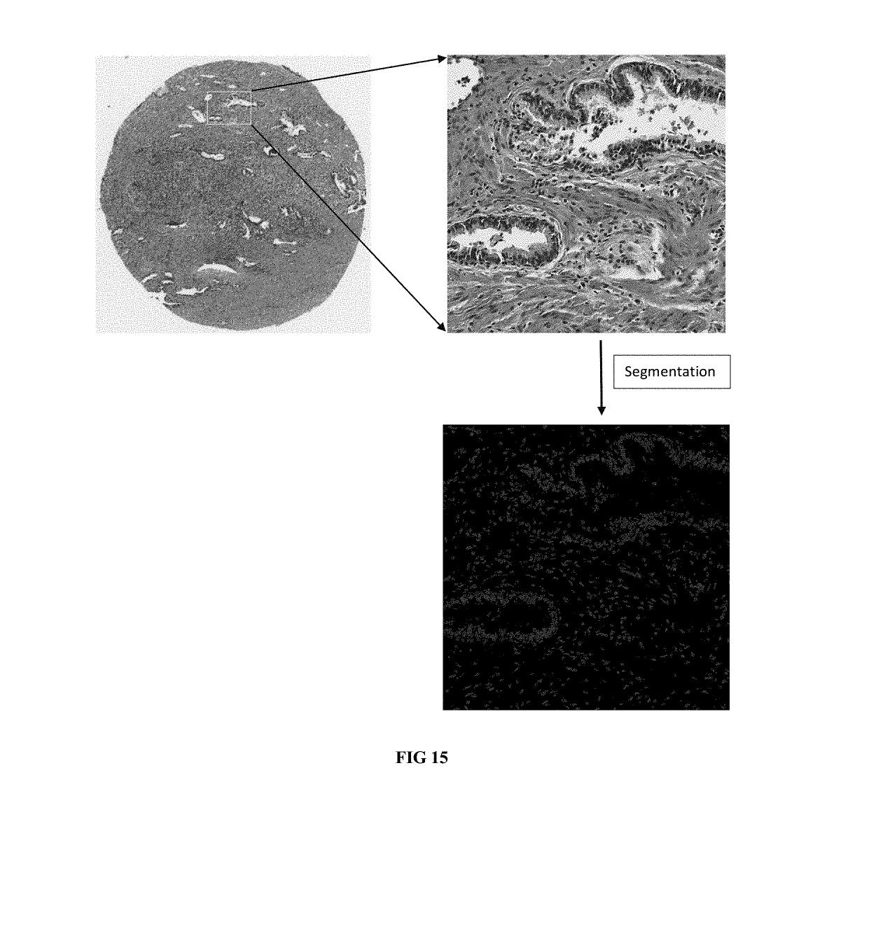

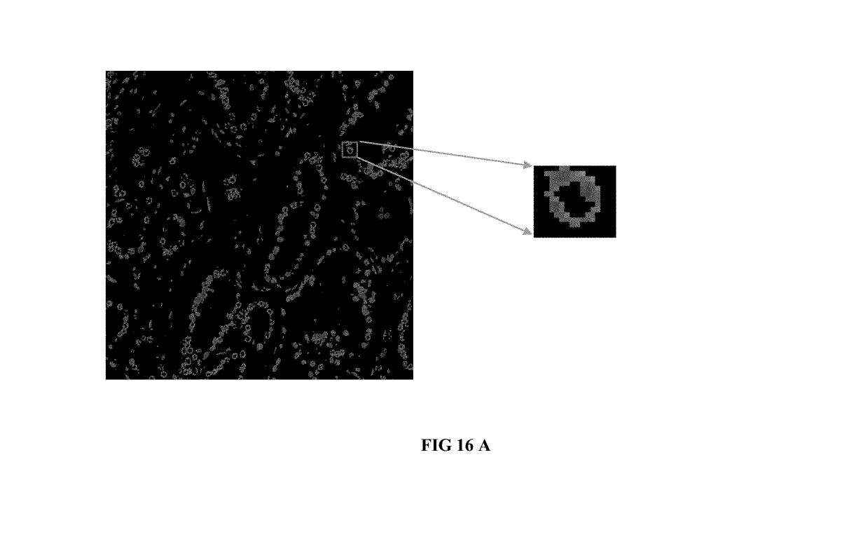

The segmented results of each color plane are fused together to form one image. FIG. 11-15 show the illustrative examples of the shape based nuclei segmentation method applied on both magnified and whole slide image.

Thresholding method is referred from Irshad, H., Veillard, A., Roux, L., & Racoceanu, D. (2014). Methods for nuclei detection, segmentation, and classification in digital needle biopsy: a review—current status and future potential. IEEE reviews in biomedical engineering, 7, 97-114.

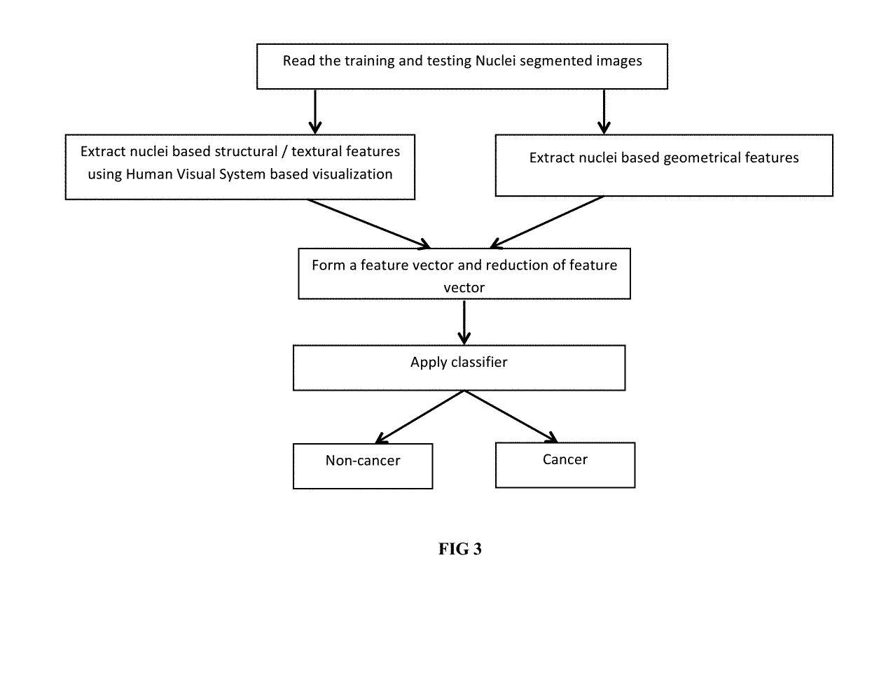

The process according to the embodiment of the present invention for cancer detection shown in FIG. 3 is described below. The first step of the process is to read nuclei segmented training and testing images as an input. The Structural and textural features are extracted from theses nuclei segmented images. These features are extracted using Human Visual System (HVS) based visualization system. The HVS based visualization system comprises of the following steps:

a. Application of the modified weber law on the image

b. Generation of the Nuclei size based Fibonacci p-patterns
a. Application of the modified weber law on the image: The images are divided into continuous M×M blocks and for every M×M block of the image, the weber coefficient for each block is calculated using the following mathematical expression of the modified weber law

$$w = \frac{1}{( \frac{N}{2} ) + 1} {\sum }_{i = 0}^{( \frac{N}{2} ) - 1} \frac{\vert {n}_{t} - {n}_{c} {\vert}^{γ}}{{n}_{t} + {n}_{c}} , t = 2 * i + 1 \qquad ( 4 )$$

Where,

N is the number of neighbors selected,

γ is the power constant,

w is the weber coefficient for block of size M×M,

nₜ is the calculated t^{th} pixel of the block, and

n_{c} is the center pixel of the M×M block.

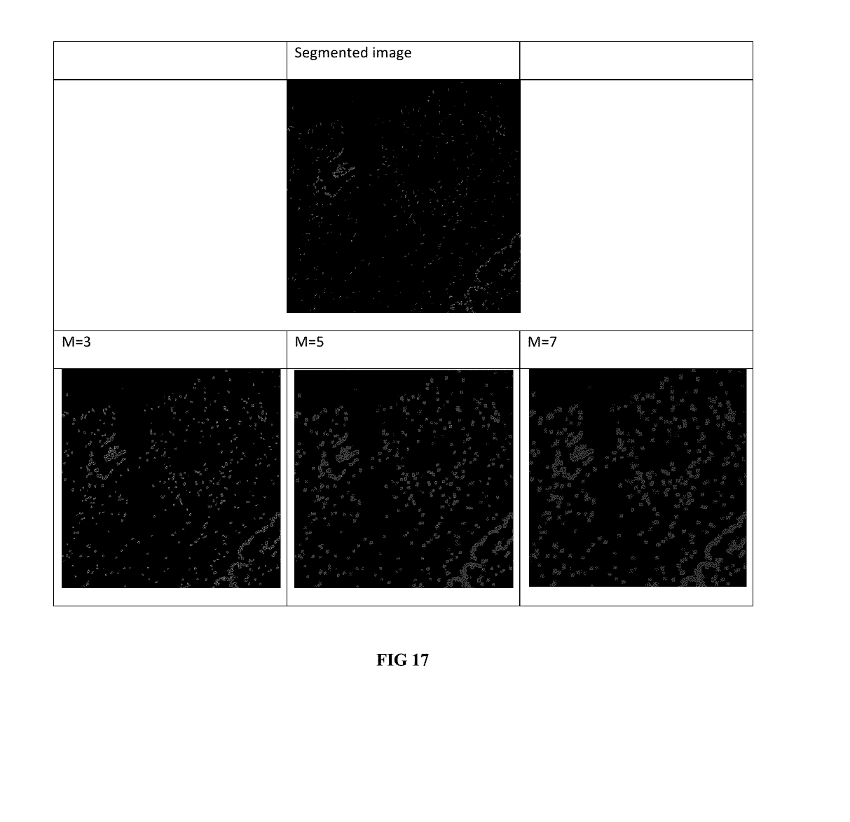

The calculated weber coefficient represents each block of the image thus forming a weber image. FIG. 17 shows the illustrative example of the weber image using different values of M.

b. Generation of the Nuclei size based Fibonacci p-patterns: Using the formed weber image, the Nuclei size based Fibonacci p-patterns are generated. Select a block of size N×N in the weber image, and divide the blocks into sub-blocks of the size m×m. The sub-blocks aligned vertically and horizontally at a radius r from the center pixel of the N×N block are selected and analysis is performed on them. The analyzed values then represent the sub-blocks. The analysis value can be evaluated using averaging, median filtering, finding maximum value or minimum value etc. A corresponding 3×3 matrix is generated such that the analyzed value is placed on the position corresponding to that of the sub-block and the center value of 3×3 block is the center pixel of the N×N block. The diagonal values of the 3×3 block are the diagonal values present in N×N block at radius r. The Fibonacci p-patterns are generated using the following mathematical equations.

$$\begin{matrix}F = {\sum }_{k = 0}^{\frac{n}{2} - 1} {b}_{k} {a}_{2 * k + 1} {f}_{p} ( k ) + {\sum }_{k = 0}^{\frac{n}{2} - 1} {c}_{k} {a}_{2 * k} {f}_{p} ( k ) \\ Where , & ( 5 ) \\ {a}_{2 * k + 1} = \{ \begin{matrix}1 , & {X}_{k} - {X}_{c} \geq t 1 \\ 0 , & {X}_{k} - {X}_{c} < t 1\end{matrix} , where k = 0 , … , \frac{n}{2} - 1 & ( 6 ) \\ {a}_{2 * k} = \{ \begin{matrix}1 , & {X}_{k} - {X}_{c} \geq t 2 \\ 0 , & {X}_{k} - {X}_{c} < t 2\end{matrix} , where k = 0 , … , \frac{n}{2} - 1 & ( 7 ) \\ {f}_{p} ( k ) = \{ \begin{matrix}0 , & k < 0 \\ 1 , & k = 0 \\ {f}_{p} ( k - 1 ) + {f}_{p} ( k - p - 1 ) , & k > 0\end{matrix} & ( 8 )\end{matrix}$$

bₖ, cₖ are the weights assigned to corresponding values of αₖ,
F is the generated Fibonacci p-patterns generated of each radius,
n is the number neighbors selected here n=8,
Xₖ is the pixel value at k^{th} place in the formed 3×3 block,
X_{c} is the center pixel of the N×N block, and

t1 and t2 are the threshold values.

αₖ, is the Fibonacci image formed for one block. In the N×N block, the values of m×m can be varied, until the N×N can be divided into disjoint m×m sub-blocks. The value of N is the average size of nuclei belonging to the particular grade of cancer. The radius r equivalent to the size of the sub-block with the center pixel of the center block as the origin. In case of magnification change, the size of N will be proportioned correspondingly for each grade. For sub-block of each size Fibonacci p-patterns are generated. Features are extracted from the obtained p-patterns are concatenated to form a feature vector giving us nuclei based structural and textural feature vector.

The Fibonacci p-patterns definition is referred from, Agaian, S., Astola, J., Egiazarian, K., & Kuosmanen, P. (1995). Decompositional methods for stack filtering using Fibonacci p-codes. Signal Processing, 41(1), 101-110.

Reference for graphical methods used are:

Nguyen, K., Sarkar, A., & Jain, A. K. (2014). Prostate cancer grading: Use of graph cut and spatial arrangement of nuclei. IEEE transactions on medical imaging, 33(12), 2254-2270.

Boucheron, L. E. (2008). Object-and spatial-level quantitative analysis of multispectral needle biopsy images for detection and characterization of cancer. University of California at Santa Barbara.

Nuclei based Geometrical features are extracted using the graphical methods. The graphical methods that can be used are Delaunay Triangulation, Spanning trees, Voronoi Tessellation etc. A Combine feature vector is generated on concatenating the obtained structural, textural and geometrical features. Feature reduction is performed using the sort and merge method. The classifier tree is applied on the reduced feature vector to classify the prostate needle biopsy images as either benign or malignant i.e. non-cancerous or cancerous.

Reference for Sort and merge method : Liu, Y., & Kender, J. R. (2003, January). Sort-Merge Feature Selection for Video Data. In SDM (pp. 321-325).

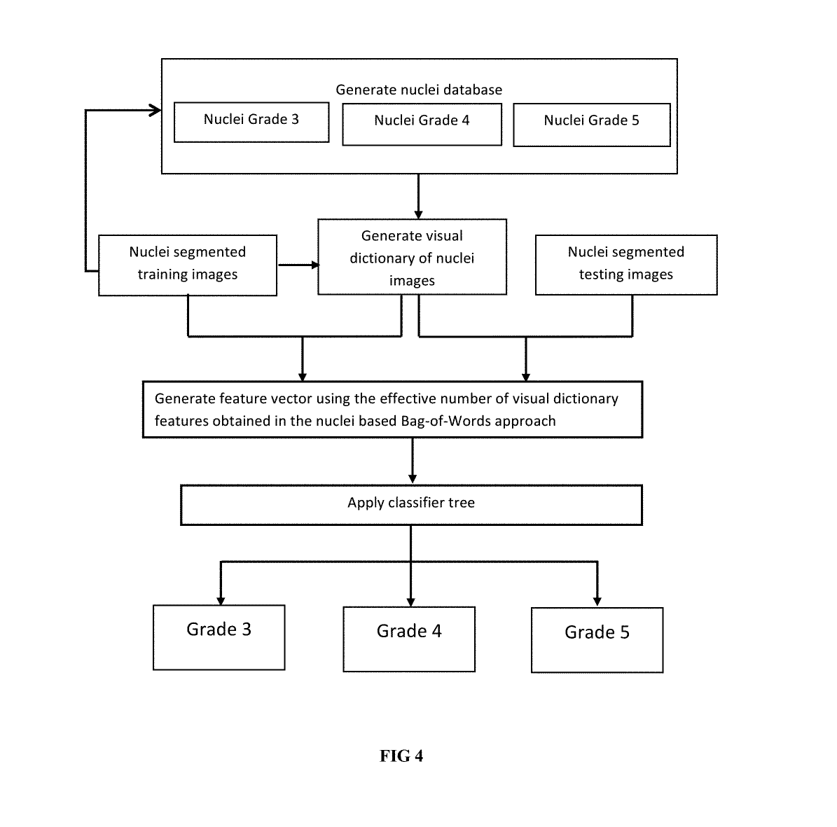

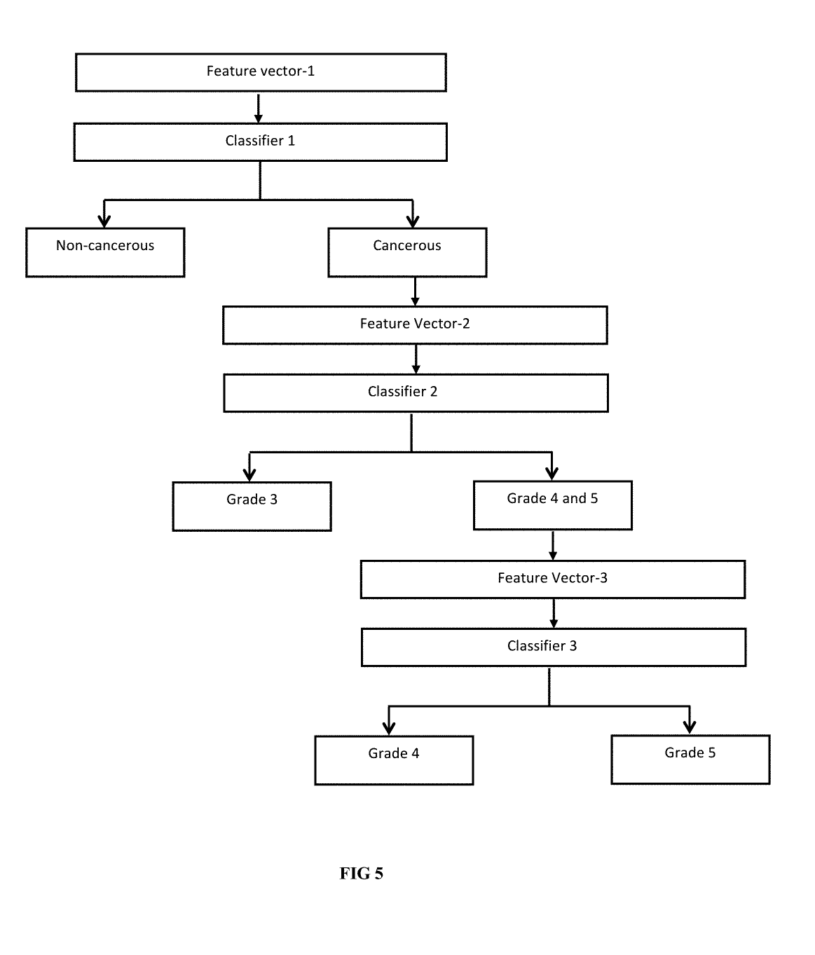

The process according to the embodiment of the present invention for classifying prostate cancer needle biopsy images into different grades shown in FIG. 4 is described below. The nuclei segmented training images are used for generating the visual dictionary of the nuclei information obtained from the extracted features. Feature vector is generated using the effective number of features extracted using the visual dictionary of images in the human interactive nuclei-based Bag-of-Words (BoW) approach. The human interactive nuclei based BoW have the following steps:

a. Feature extraction

b. Formation of visual dictionary of nuclei information

c. Quantizing visual dictionary features and generating probability response of visual words present in each segmented image.

a. Feature extraction: The Speeded-UP Robust Feature(SURF) and nuclei density features are extracted from the training and testing nuclei segmented images. The SURF feature descriptors provide the nuclei locations in the image which are further used for extracting the features from the nuclei. The nuclei density feature comprises of the concentration of nuclei present in other tissue components such as cytoplasm, stroma, or lumen.

b. Formation of the visual dictionary of nuclei information: SURF and nuclei features of few training images are utilized to form the visual dictionary of nuclei information. To generate the dictionary, the features are clustered using clustering method. The number of clusters used in clustering method is equivalent to the number of words to be employed in visual dictionary, which are further used to compute the probability presence of each word in each grade of cancer. The cluster centers obtained from the clustering algorithm represent words of each grade cancer present in the visual dictionary. Along with the words obtained using SURF and nuclei feature, manually generated nuclei database features is also combined before the clustering algorithm. Thus, the cluster centers in the dictionary of words consist both the manually and automated generated visual word.

c. Quantizing visual dictionary features and generating probability response of visual words present in each segmented image: The dictionary contains the nuclei information for grade 3, 4, and 5, however it is necessary to ensure the word belongs to the appropriate cancer grade. To achieve this, the probability responses of visual words present in each segmented image are calculated and histograms are generated. To generate the probability response, the nearest words to the feature are found using the distance metrics. Using the nearest distances and the cluster centers, probability responses i.e. histograms of the words for each image in each grade are generated. Higher the probability, higher are the chances of the word belonging to the particular cancer grade.

The bag of words approach is referred from, Masterravi. (2011 Mar. 17). Object recognition using bag of features. Retrieved Sep. 8, 2016, from https://masterravi.wordpress.com/2011/03/17/object-recognition-using-bag-of-features/.

The reference for SURF is Bay, H., Ess, A., Tuytelaars, T., & Van Gool, L. (2008). Speeded-up robust features (SURF). Computer vision and image understanding, 110(3), 346-359.

The distance metrics is referred from:

Huang, A. (2008, April). Similarity measures for text document clustering. In Proceedings of the sixth new zealand computer science research student conference (NZCSRSC2008), Christchurch, New Zealand (pp. 49-56).

ClassificationKNN. (1994). Classification using nearest neighbors. Retrieved Sep. 8, 2016, from http://www.mathworks.com/help/stats/classification-using-nearest-neighbors.html

For identifying grade 3 cancer images from grade 4 and 5, and distinguishing between grade 4 and 5 different number of clusters are used, thus two separate feature vectors are generated. Feature reduction is performed to achieve effective number of features. The classifier tree is applied for classifying the prostate cancer needle biopsy images into different grades of cancer i.e. Grade 3, 4 and 5.

The process according to the embodiment of the present invention for the decision tree classifier ensemble shown in FIG. 5 is described below. The feature vector-1 generated from selected concatenated structural, textural and geometrical nuclei based features is classified using a one versus all approach classifier. In this stage, the training feature vector is used for training the classifier, which is used for classifying the testing images as either cancerous or non-cancerous using testing feature vector. The detected cancerous images are further used for detecting the grade of cancer. Gleason grade 3 cancer images are first identified and separated from the higher cancer grades viz. grade 4 and 5. To identify grade 3 cancer, the nuclei-based BoW features are extracted. The features are classified either as grade 3 or as higher grades of cancer using the one-versus all approach classifier. Nuclei-based BoW based features are extracted from the training and detected images of higher cancer grades visualizing grade 4 and 5. The detected higher cancer grade images are classified as either grade 4 and 5 using the one versus one approach classifier. For the decision tree classifier any classification method can be used for this purpose, SVM, K-NN classify, Naïve Bayes, Discriminant analysis, etc.

The classification methods are referred from Kiang, M. Y. (2003). A comparative assessment of classification methods. Decision Support Systems, 35(4), 441-454.

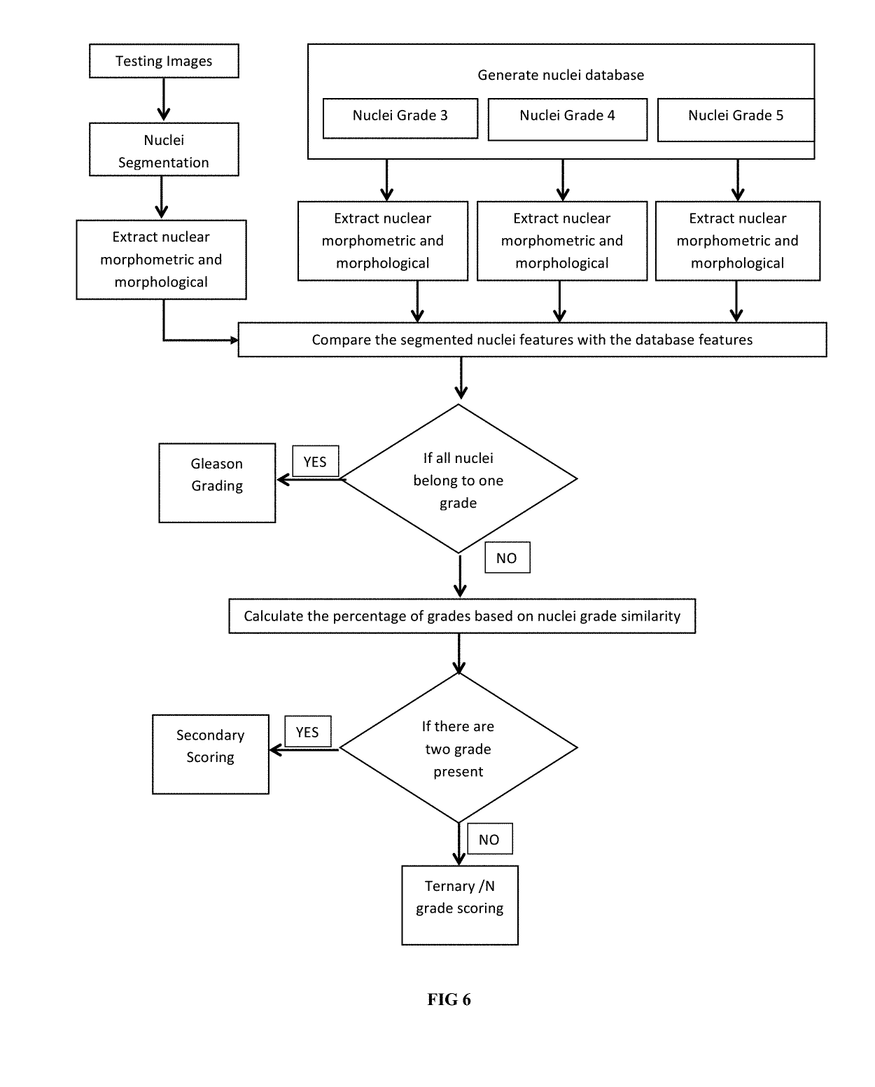

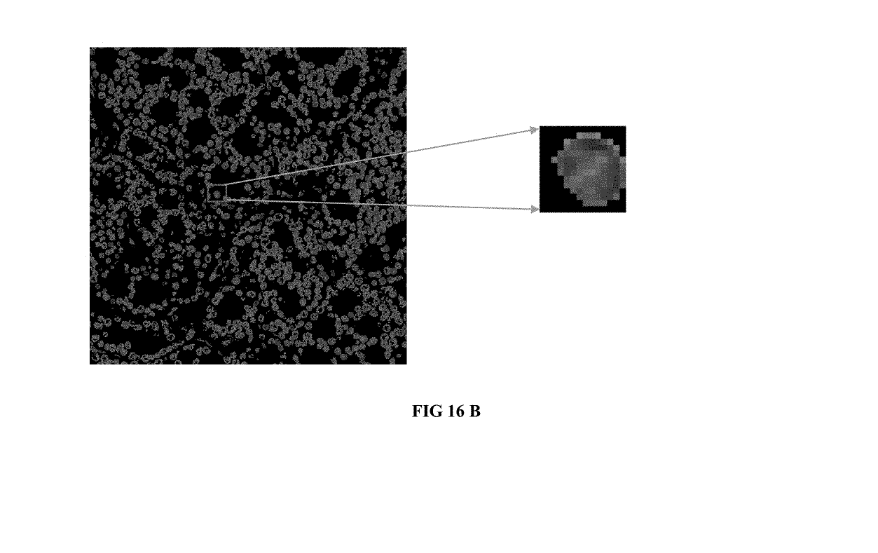

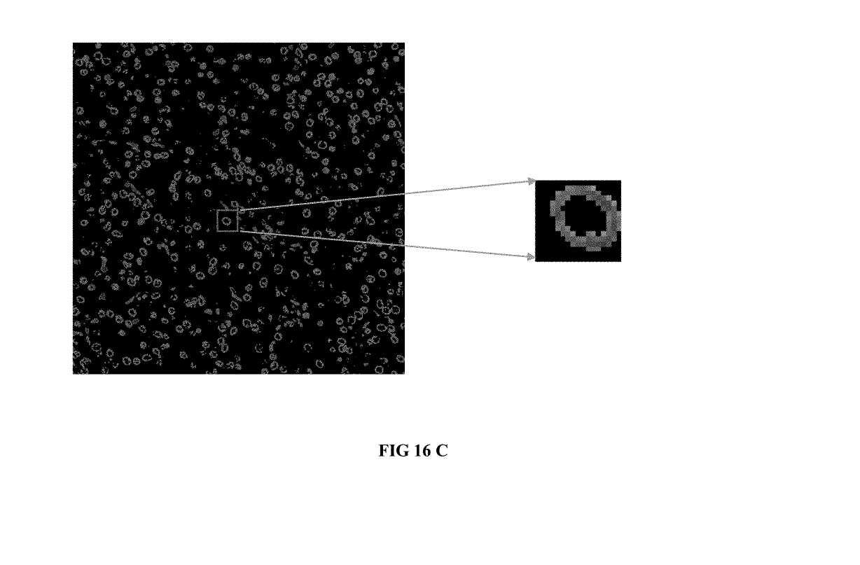

The process according to the embodiment of the present invention for the Gleason scoring approach for the whole slide prostate cancer needle biopsy images using nuclei morphometric features shown in FIG. 6 is described below. A nuclei database is generated for Grade 3, 4, and 5 using the training images. The illustrative examples of the nuclei for grade 3,4 and 5 is shown in FIG. 16A-C. The testing images are read and nuclei segmentation is performed on them. The morphometric and morphological features are extracted from both, the nuclei database and the segmented testing whole slide images. The different morphological features that can be extracted are size, shape, area, presence of nucleoli etc. Features extracted from each nucleus of each cancer grade of the database are compared to the nuclei features present in the testing image by computing similarity measurement. If all the nuclei in the testing image is similar to the one cancer grade then primary Gleason grading is assigned to them, or else the percentage of each cancer grade present is calculated. If two grades are present then secondary scoring is assigned to the image following the rules of secondary Gleason scoring given by the American cancer society, or else ternary or N-Gleason scoring is assigned depending on the number of grades present.

Similarity measurements: Goshtasby, A. A. (2012). Similarity and dissimilarity measures. In Image registration (pp. 7-66). Springer London.

Gleason scoring guidelines: Prostate Cancer. Retrieved Sep. 9, 2016, from American Cancer Society, http://www.cancer.org/acs/groups/cid/documents/webcontent/003134-pdf.pdf

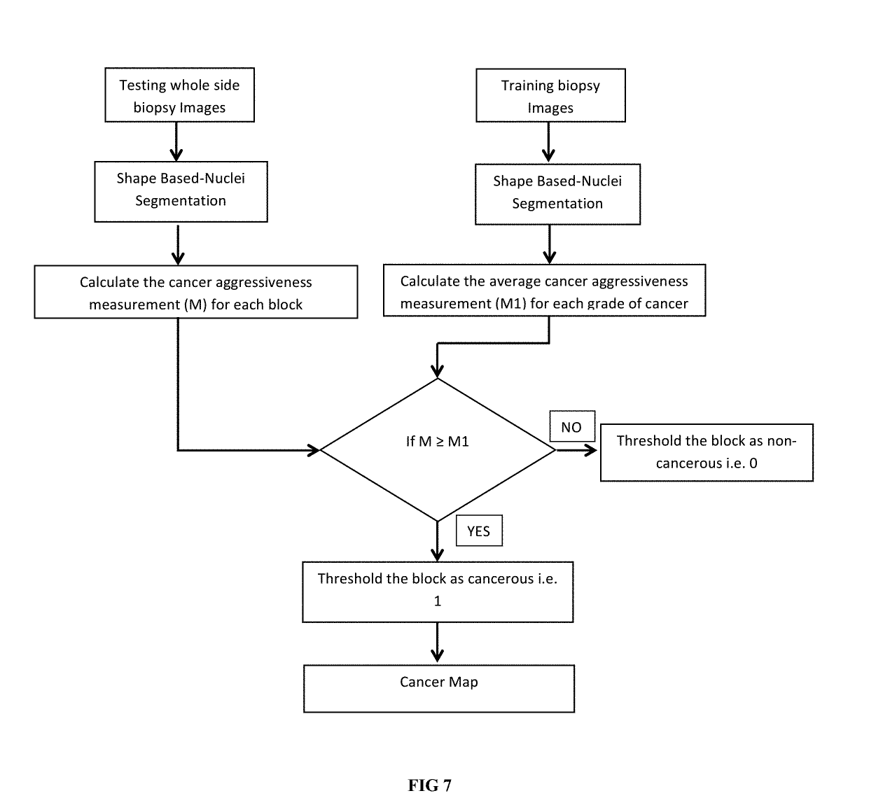

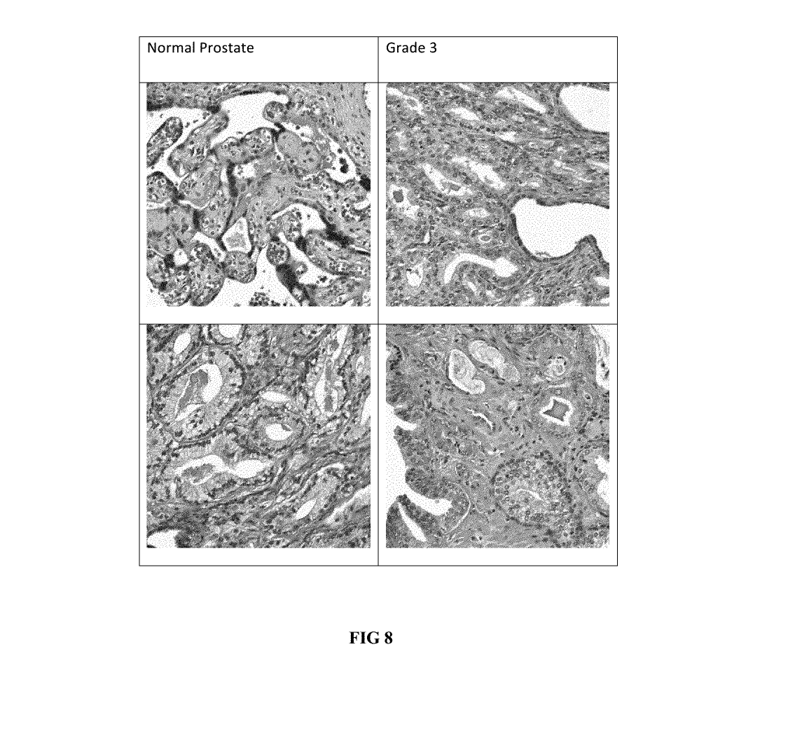

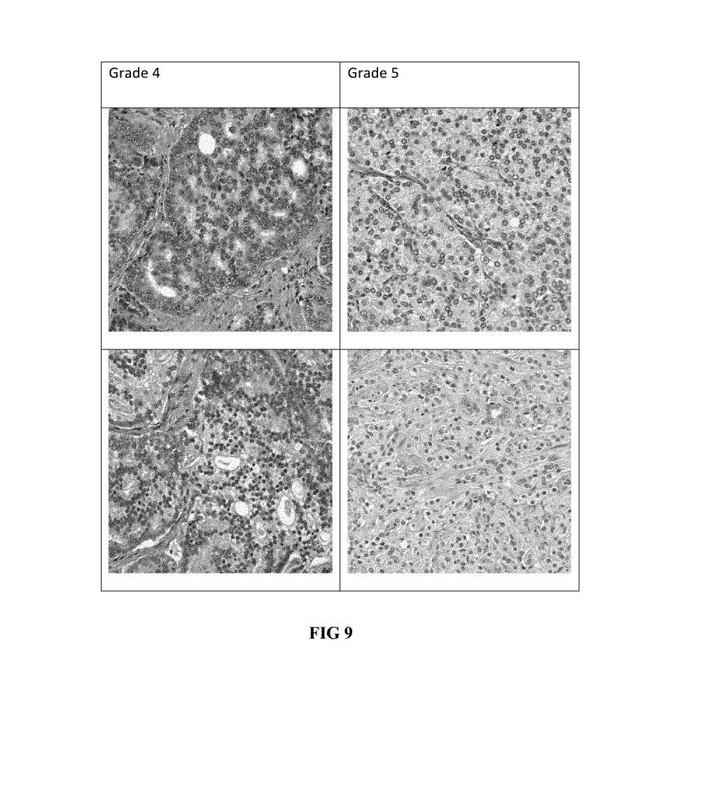

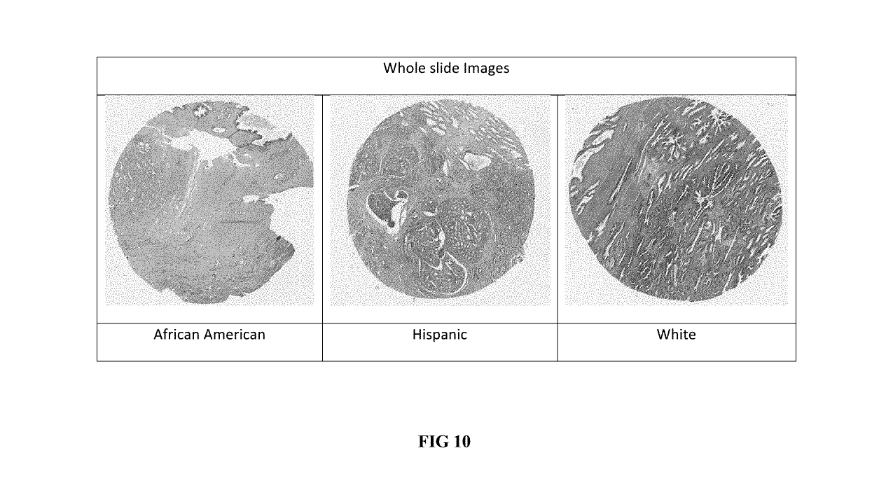

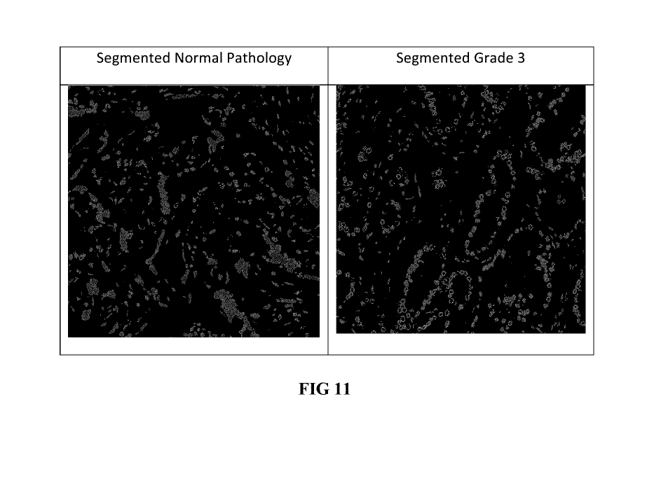

The process according to the embodiment of the present invention for Cancer Map generation using the cancer aggressive measurement features shown in FIG. 7 is described below. Training magnified images and testing whole slide images are taken as an input and shape based nuclei segmentation is applied on them. For each segmented block obtained, cancer aggressiveness measurement (CAM) is computed on each block using the following mathematical equation:

$$CAM ( M ) = \frac{N - R}{N + R} \qquad ( 9 )$$

Where,

M=CAM calculated for the segmented block image of whole slide prostate biopsy image.

N=Nuclear area calculated in the image

R=Residual area calculated in the image

For training images, the average cancer aggressiveness measurement for each cancer grade is calculated using the following mathematical equation:

$$CAM ( M 1 ) = \frac{1}{k} {\sum }_{i = 1}^{k} \frac{{N}_{k} - {R}_{k}}{{N}_{k} + {R}_{k}} \qquad ( 10 )$$

Where,

M1=averaged calculated CAM from the training images,

Nₖ=Nuclear area calculated for the k^{th} cancer image,

Rₖ=Residual area calculated for the k^{th} cancer image, and

K is the number of the cancer images.

If the measurement of the testing image block (M) is greater than or equal to the measurement of the training images (M1), the block is thresholded as cancerous and is given a value 1, or else the block is thresholded as non-cancerous and given value 0. Thus, helping in generating a cancer map of the image.

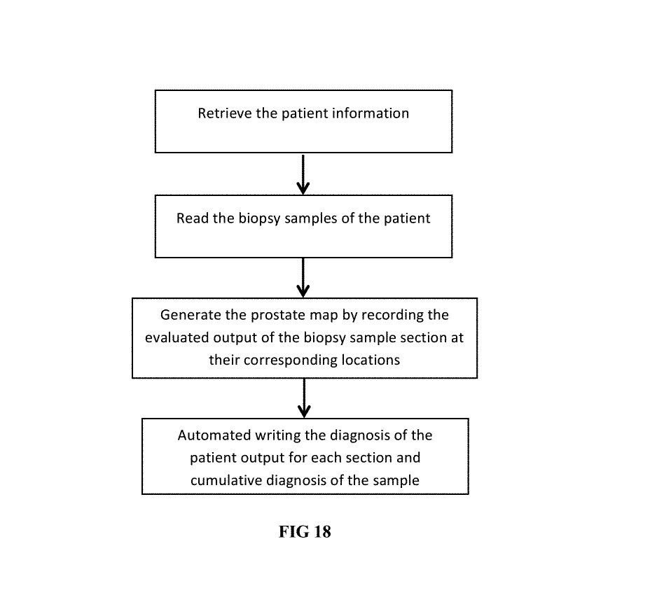

The process according to the embodiment of the present invention automated report generation system shown in FIG. 18 is described below. The patient information is retrieved from the database entered by the pathologist before conducting the biopsy. The patient information includes, the name, age, date of birth, status, race, family history, PSA levels, cancer recurrence, the pathologist's information etc. After retrieving the patient data, the data is auto-written in the report. The prostate map is generated indicating the sections of biopsy sample. The sections are placed at their corresponding anatomical location from where they are extracted. The sections of the biopsy samples are analyzed and their prognosis is written on the prostate map. A cumulative diagnosis of the entire biopsy sample is prepared and is auto-written. Thus a complete automated report is generated.

Further modifications and alternative embodiments of various aspects of the invention will be apparent to those skilled in the art in view of this description. Accordingly, this description is to be construed as illustrative only and is for the purpose of teaching those skilled in the art the general manner of carrying out the invention. It is to be understood that the forms of the invention shown and described herein are to be taken as examples of embodiments. Elements and materials may be substituted for those illustrated and described herein, parts and processes may be reversed, and certain features of the invention may be utilized independently, all as would be apparent to one skilled in the art after having the benefit of this description of the invention. Changes may be made in the elements described herein without departing from the spirit and scope of the invention as described in the following claims.

What is claimed is:

1. A system and a method for diagnosing and classifying the Prostate Cancer Needle biopsy Images comprising:
collecting training data and testing data, wherein testing data has whole slide images and magnified images;
segmenting the nuclei information from the other tissue components using the alpha-trimmed mean;
generating feature vector using the nuclei information to diagnose cancer in the prostate needle biopsy images;
classifying the obtained feature vector through the decision tree classifier to diagnose cancer;
generating visual dictionary of nuclei images only if the images are detected cancerous;
extracting features using the visual dictionary and generating feature vector;
classifying the cancer images into their respective grades using the decision tree classifier.

2. A method for shape based segmenting of the nuclei information from the images comprising:
dividing the images into block of the size of the training images if the images are whole slide images or else proceed to next step of image enhancement;
performing image enhancement;
selecting a color model and separating the image into different color planes of the selected model;
performing segmentation using threshold (T) based on Alpha-trimmed mean on each color planes using the following formulae.
$$t = \frac{1}{MN} {\sum }_{i = 0}^{M - 1} {\sum }_{j = 0}^{N - 1} [ ( \frac{a}{a + b} ) I ( i , j ) - ( \frac{b}{a + b} ) α ( i , j ) ]$$
$$T = max ( ∝ ) + t$$
Where,
α is the alpha-trimmed mean image of the image I,
I is the color plane of the image,
a, b is the constants,
M, N is the size of the image,
t is the mathematical function calculating weighted difference between the image I and α-trimmed mean image for each pixel, and
T is the threshold value calculated by taking sum of the maximum value of a trimmed mean and t.
fusing the obtained segmented images to form a color segmented image;

3. A method for diagnosing cancerous and non-cancerous prostate needle biopsy images comprising:
reading the nuclei segmented images of training and testing database;
extracting nuclei based structural/textural features using Human Visual System based visualization;
extracting nuclei based geometrical features using graphical methods;
generating feature vector on combining features obtained and performing feature selection;
training the decision tree classifier and classifying the test data set using the trained classifier as either cancerous or non-cancerous.

4. A method for Human Visual System based visualization comprising:
reading the shape based nuclei segmented image;
generating weber image using the modified weber law giving by the formula
$$w = \frac{1}{( \frac{N}{2} ) + 1} {\sum }_{i = 0}^{( \frac{N}{2} ) - 1} \frac{\vert {n}_{t} - {n}_{c} {\vert}^{γ}}{{n}_{t} + {n}_{c}} , t = 2 * i + 1$$
Where,
N is the number of neighbors selected,
γ is the power constant,
w is the weber coefficient for block of size M×M,
nₜ is the calculated t^{th} pixel of the block, and
n_{c} is the center pixel of the M×M block.
selecting a N×N block from the weber image and dividing into sub-blocks of size m×m;
analyzing the horizontally and vertically aligned sub-blocks with respect to center sub-block;
generating a 3×3 block such that above obtained analyzed value is stored in their corresponding locations and diagonal values as those obtained at radius r from the center pixel in the center block;
generating the nuclei-structured based Fibonacci p-patterns using the following mathematical equation:
$$F = {\sum }_{k = 0}^{\frac{n}{2} - 1} {b}_{k} {a}_{2 * k + 1} {f}_{p} ( k ) + {\sum }_{k = 0}^{\frac{n}{2} - 1} {c}_{k} {a}_{2 * k} {f}_{p} ( k )$$
$$Where , \\ {a}_{2 * k + 1} = \{ \begin{matrix}1 , & {X}_{k} - {X}_{c} \geq t 1 \\ 0 , & {X}_{k} - {X}_{c} < t 1\end{matrix} , where k = 0 , … , \frac{n}{2} - 1 \\ {a}_{2 * k} = \{ \begin{matrix}1 , & {X}_{k} - {X}_{c} \geq t 2 \\ 0 , & {X}_{k} - {X}_{c} < t 2\end{matrix} , where k = 0 , … , \frac{n}{2} - 1 \\ {f}_{p} ( k ) = \{ \begin{matrix}0 , & k < 0 \\ 1 , & k = 0 \\ {f}_{p} ( k - 1 ) + {f}_{p} ( k - p - 1 ) , & k > 0\end{matrix}$$
bₖ, cₖ are the weights assigned to corresponding values of αₖ,
F is the generated Fibonacci p-patterns generated of each radius,
n is the number neighbors selected here n=8,
Xₖ is the pixel value at k^{th} place in the formed 3×3 block,
X_{c} is the center pixel of the N×N block,
t1 and t2 are the threshold values, and
αₖ, is the Fibonacci image.

5. A method for identifying the grade of cancer for the detected cancerous prostate needle biopsy images comprising:
reading the nuclei segmented images of training and testing set;
generating manually the nuclei database for different grades of cancer;
generating dictionary of visual words using the automated and manually generated nuclei segmented training set of images;
generating feature vector using the effective number of visual dictionary features obtained in the human interactive nuclei based Bag-of-Words approach;
applying classifier tree to classify the testing set of cancerous needle biopsy images to its respective grade of cancer i.e. Grade 3, 4 or 5.

6. A method of decision tree based classifier ensemble comprising:
reading the feature vector-1 generated from the nuclei based structural and geometrical features;
applying classifier 1 for detecting cancerous and non-cancerous prostate needle biopsy images;
reading feature vector 2generated from the nuclei based bag-of-words approach;
applying classifier 2 for classifying the Gleason grade 3 prostate cancer needle biopsy images from grade 4 and 5;
reading feature vector 3 generated from the nuclei based bag-of-words approach;
applying classifier 3 for classifying the Gleason grade 4 prostate cancer needle biopsy images from grade 5.

7. A system and method for Gleason scoring the prostate cancer needle biopsy images comprising:
generating nuclei database for each Gleason grade 3,4 and 5;
reading the testing set of images;
performing nuclei segmentation on the testing images;
extracting nuclear morphometric features from both, the nuclei database and testing images;
comparing the segmented nuclei features with the generated database features;
assigning primary grading if the nuclei in the testing image belong to one grade of cancer;
calculating the percentage of grades present in the testing image if there are more grades present based on nuclei grade similarity;
assigning secondary scoring if percentage similarity of two grades are present in the testing images or else ternary or n-scoring if 3 or more grades are counted for testing image.

8. A system or method of generating a cancer map using the cancer aggressiveness measurement comprising:
reading the test whole slide images and train magnified set of images;
performing nuclei segmentation on both training and testing data set images;
calculating the cancer aggressiveness measurement for each block of the segmented whole slide image;
calculating the average cancer aggressiveness measurement for grade of cancer from the training images;
assigning the block value 1 if the value of cancer aggressiveness measurement for testing block is greater or equal then training block or else 0;
generating cancer map from the above step.

9. A system of automated report generation comprising:
retrieving the patient information;
reading the different sections of the biopsy sample;
generating the prostate map by recording the evaluated output of the biopsy sample sections at their corresponding location;
automated writing of the diagnosis of the patients' output for each biopsy section of the sample and cumulative diagnosis of the sample.
